(19) United States
(12) Patent Application Publication
(10) Pub. No.: US 2019/0206566 A1
(43) Pub. Date: Jul. 4, 2019

(54) MOVEMENT DISORDERS MONITORING AND TREATMENT SUPPORT SYSTEM FOR ELDERLY CARE

(71) Applicant: VOCATIONAL TRAINING COUNCIL, Hong Kong (HK)

(72) Inventors: Raymond Kwan LAI, Hong Kong (HK); Ivan Man Chim TSE, Hong Kong (HK); Calvin Hoi Kok CHEUNG, Hong Kong (HK); Timothy Kin Sang LEE, Hong Kong (HK); Chi Pang LAM, Hong Kong (HK); Sing Yee NG, Hong Kong (HK); Eric Sai Lok LIU, Hong Kong (HK); Ching Ching CHEUNG, Hong Kong (HK); Leung CHIU, Hong Kong (HK); Hon Kong CHAN, Hong Kong (HK); Hung Keung TSE, Hong Kong (HK)

(21) Appl. No.: 15/856,888

(22) Filed: Dec. 28, 2017

(51) Int. Cl. G16H 40/67; A61B 5/00; A61B 5/11; G16H 20/10; G16H 50/30; G16H 15/00

(52) U.S. Cl. CPC G16H 40/67; A61B 5/0022; A61B 5/1124; A61B 5/1101; G06Q 10/1097; G16H 20/10; G16H 50/30; G16H 15/00; A61B 5/4082

(57) ABSTRACT

Disclosed is a monitoring and treatment support system to monitor motion symptoms of tremor, bradykinesia and/or dyskinesia. A system and method are also provided for early detection of movement disorders. Further, a system and method are provided which can accurately quantify symptoms utilizing at least one measuring device at a scheduled time as arranged by medical professionals. The timer in the digital diary will remind the elderly to take medications and/or to perform motion tests. A system and method are also provided which can compute an overall motor performance score using weighting algorithm according to the results of tremor test, finger tapping test and/or spiral drawing test. The overall motor performance score is presented using comprehensive figures to both medical professionals and the elderly as a summary report for their review. The severity of movement disorders presented in graphs is compared with the treatment plan for analysis.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

### Field of Invention

The present invention relates to a movement disorder monitoring and treatment support system, and a method of measuring and quantifying symptoms of movement disorders. More particularly, the present invention relates to a monitoring and treatment support system to monitor motion symptoms of tremor, bradykinesia and/or dyskinesia.

### Description of Related Art

Movement disorders include, but are not limited to, Parkinson's disease (PD), dystonia, Tourette's syndrome and essential tremor. People with movement disorders usually present syndromes with an excess of movement or a paucity of voluntary and involuntary movements. The symptoms include tremor, bradykinesia, rigidity, dyskinesia, and the like. Take Parkinson's disease as an example, tremor, bradykinesia and dyskinesia are some of the major symptoms that affect the quality of life of the elderly. Movement disorders are especially prevalent in the elderly. The prevalence of all common categories of movement disorders in men and women aged 50-89 years was 28%, and the proportions were sharply increased with age.

Tremors are unintentional and uncontrollable rhythmic movements of the body part of the elderly. They are the result of a problem in part of the brain that controls muscular movement. Tremors can occur in any part of the body at any time. Bradykinesia is characterized by slow movement and an impaired ability to move the body swiftly on commands. It is the most common symptom of Parkinson's disease. Dyskinesia is a kind of involuntary movements. The severity of dyskinesia differs with the elderly, affecting normal daily activities of the elderly. Its symptoms can occur at any frequency or at any time of day.

To monitor and track the frequency and the severity that the elderly suffered from symptoms of movement disorders, a diary is mainly used as a tool to monitor the symptoms of movement disorders at present. However, a traditional diary relies heavily on the elderly's motivations and self-discipline to fill out the diary. This kind of self-report is subjected to bias that the elderly who are not medical professionals may not be able to distinguish one symptom from other symptoms. The reliability of the traditional diary would also be hindered due to the subjective experience and subjective observation by the elderlies. Moreover, the traditional diary requires only filling out the ON and OFF states of the symptoms. The ON and OFF states are inadequate for measuring and monitoring the severity of the symptoms. As the result, records from the traditional diary may not be sufficient for medical professionals, include but are not limited to, a physician and clinician, when assessing the severity of movement disorders and making important clinical decisions such as writing up and adjusting prescriptions.

Recently, some efforts have been made to quantify symptoms of movement disorders using digitized monitoring system such as a digital diary to replace the use of a traditional handwritten diary to refrain from the problems arising from the elderly's motivations and self-discipline. However, these digitized monitoring systems require continuous monitoring of movement disorders during everyday activities, over a 24-hour period. The continuous monitoring during daily activities risks the possibility of containing errors in the measurement. The movements in daily activities are easily messed up with motions of movement disorders. Although there are prior arts suggested methods for distinguishing symptoms of movement disorders from activities of daily living, errors occurred in the measurement during activities can never be eliminated. Also, the day and night wearing of the measuring device throughout the day put the elderly to great inconvenience.

Moreover, the existing digitized monitoring and measuring systems are designed to quantify symptoms of movement disorders individually. The digitized monitoring and measuring system processes and analyzes data from the elderly and hence computes various scores separately. Each individual score is an indicator of one symptom of movement disorders. For example, the existing digitized monitoring system obtains data from the elderly and utilizes the captured data to compute one score for tremor symptom, one score for bradykinesia symptom and/or one score for dyskinesia symptom. These individual scores of various symptoms are presented to medical professionals and the elderly to have a review. However, providing scores of various symptoms of movement disorders exclusively may create difficulty for the elderly to understand their own health conditions and to know more about the status of their treatment in the home environment. The nonlinearity in inter- and intra-test score becomes the main difficulty to interpret the treatment status without medical professionals. It may also influence the judgment and the clinical decisions made by a medical professional. Medical professionals may solely focus on one symptom of movement disorders while neglecting the general situation of the elderly when assessing the severity of movement disorders and making up a prescription for the elderly. Medications for treating movement disorders are not given depending on only a symptom of movement disorders. Medical professionals should take an overview and understand the general conditions of the elderly when prescribing them medications. Movement disorders are characterized by ranges of symptoms include, but are not limited to, tremor, bradykinesia, rigidity and/or dyskinesia. Suffering from severe tremor does not necessarily means that the elderly are having a severe movement disorder. To make precise judgements when assessing the severity of movement disorders, a whole picture should be presented to medical professionals by integrating the results of various symptoms of movement disorders.

Furthermore, the existing digitized monitoring and measuring systems concentrate their design merely on the method of carrying out the measurement, while paying less attention to the target users of the invention. As a monitoring and measuring system for reference to medical professionals, the invention should put more emphasis on the elderlies themselves. Therefore, it is important to have a personalized system for monitoring and measuring symptoms of movement disorders.

More importantly, the existing digitized monitoring and measuring systems are developed for the elderlies already diagnosed with movement disorders to have further monitoring in the home environment to facilitate treatment procedures by medical professionals. However, providing systems only for those who have already diagnosed with movement disorders is not the most effective way to treat movement disorders. Instead, a system should be provided to the general public, even the elderly or persons without movement disorders, to allow regular monitoring and early detection of movement disorders of the elderly or persons.

It is therefore an object of the present invention to provide a system for quantifying accurately symptoms of movement disorders. It is another object of the present invention to provide a system that quantifies symptoms accurately utilizing measuring devices which are easy to use for the elderly and convenient enough for home monitoring. It is another object of the present invention to provide a system that allows early detection of movement disorders. It is still another object of the present invention to provide a system that can be worn and/or used upon reminders to monitor and quantify symptoms and provide information to be analyzed as needed by medical professionals include, but are not limited to, a physician and clinician. It is further another object of the present invention to provide a system that is capable of automatically and immediately retrieving data from measuring devices and providing the data for further analysis and evaluation. It is still further another object of the present invention to provide a system that is capable of computing an overall motor performance score from records derived from various motion tests of symptoms of movement disorders for medical professionals. It is still further an object of the present invention to provide a system with clinical video instruction to act as a guide for the elderly in home monitoring. It is further an object of the present invention to provide a personalized system for the elderly to monitor and quantify symptoms of movement disorders so that medical professionals can give directions specifically to the elderly to accommodate conditions of different elder persons. Finally, it is the object of the present invention to provide remote access to medical professionals for quantifying, monitoring and recording the motion-related symptoms of the elderly with movement disorders.

### BRIEF SUMMARY OF THE INVENTION

The present invention relates to a system and method for monitoring and quantifying symptoms of movement disorders. The present invention overcomes the shortcomings in technology to provide a system and method which can monitor and quantify multiple symptoms of movement disorders at a scheduled time as assigned by medical professionals. Medical professionals arrange personalized schedules for the elderly to accommodate conditions of different elder persons. A timer in the mobile application alerts the elderly at the scheduled time. Therefore, the elderly do not have to wear or carry the measuring devices over 24 hours. The present invention further provides a system and method for early detection of movement disorders.

The present invention still further provides a system and method for providing data from multiple measuring devices to be analyzed as needed by medical professionals, determining the severity of the elderly's movement disorders. The present invention yet further provides a system and method for providing the captured movement data from measuring devices to be computed as an overall motor performance score by scores from tremor test, finger tapping test and/or spiral drawing test using a weighting algorithm. Medical professionals can then refer to the overall motor performance score to have an overview of the general conditions of the elderly's movement disorders, instead of focusing entirely on one symptom of movement disorders.

The present invention further provides a system and method for presenting the overall motor performance score in form of comprehensive figures as needed by medical professionals for their review and analysis. The overall motor performance score is presented in graphs, and it is compared with the treatment plan to the elderly, and hence supporting medical professionals in the treatment process such as writing up and/or adjusting prescriptions.

### BRIEF DESCRIPTION OF THE DRAWINGS

FIG. 1 is a flowchart illustration showing a method for monitoring and quantifying movement disorders in accordance with one embodiment of the present invention.

FIG. 2 is a block diagram showing the components used in the tremor test.

FIG. 3 is a detailed diagram of basic components and interconnections of an embodiment of the external sensor module.

FIG. 4 is a flowchart illustration showing a method for monitoring and quantifying the performance of the elderly in a tremor test.

FIG. 5 is a system diagram showing the interconnections between each component when conducting a tremor test

FIG. 6 is a flowchart illustration showing a method for monitoring and quantifying the performance of the elderly in a finger tapping test.

FIG. 7 is a block diagram showing the interconnections between each component when conducting a finger tapping test

FIG. 8 is a diagram illustrating a user interface of the finger tapping test

FIG. 9 is a flowchart illustration showing a method for monitoring the performance of the elderly using a spiral drawing test.

FIG. 10 is a block diagram showing the interconnections between each component when conducting a spiral drawing test

FIG. 11 is a diagram illustrating a user interface of the spiral drawing test.

FIG. 12 is a flowchart illustration showing the calculation of the weighting algorithm.

FIG. 13 is a diagram illustrating a user interface of the summary report generated in the digital diary.

### DETAILED DESCRIPTION OF THE INVENTION

The present invention relates to a movement disorder monitoring and treatment support system, and a method of measuring and quantifying symptoms of movement disorders. The present invention additionally relates to a monitoring and treatment support system to monitor motion symptoms of tremor, bradykinesia and/or dyskinesia. The devices, systems and methods of the various embodiments of the present invention are used to quantify, analyze and score various symptoms of movement disorders. Movement disorders in the present application, include but are not limited to, Parkinson's disease (PD).

The devices of the various embodiments of the present invention are preferably portable. Further, it is relatively easy for the device to be carried by a single elder person. Furthermore, the device preferably should be relatively light-weight. By being light-weight, the device should gain greater acceptance for use by the elderly.

Another advantage of the system and method of the present invention is the ability to monitor and track the severity of the elderly's symptoms of movement disorders accurately without the necessity of continuous monitoring. Monitoring symptoms of movement disorders of the elderly continuously would decrease the accuracy of the measurement due to the confusion between symptoms of movement disorders and activities of daily living. Daily activities include but are not limited to handwriting, eating, dressing, and the like. To eliminate errors made during continuous monitoring, the present invention preferably involves a timer in a digital diary to perform a reminder function to remind the elderly at the scheduled time. The time is scheduled by medical professionals depending on the health conditions of the elderly. With regular measurement on symptoms of movement disorders, a medical professional could maintain the consistency of the movement data collected and ensure the accuracy of the measurement in a more convenient way without carrying bulky external measuring devices include but are not limited to external sensor modules 200 at all time.

Another advantage of the system and method of the present invention is the ability to have early detection of movement disorders. Movement abnormality of the elderly is detected and quantified to compare with the healthy elderlies to provide an advantage of detecting movement disorders in an earlier stage.

Another advantage of the system and method of the present invention is the ability to determine and calculate the severity of the elderly's movement disorders. Preferably, two or more symptoms are quantified. The symptoms include but are not limited to tremor, bradykinesia and dyskinesia. The results of the two or more symptoms are weighted using a weighting algorithm 116 and computed as an overall motor performance score 118 for medical professionals to take references and determine the severity of the elderly's movement disorders.

The devices of the various embodiments of the present invention can form part of a system for use by a medical professional, includes but are not limited to, a physician and a clinician for analyzing and assessing of the elderly's movement disorders. Other elements of the present system may include but are not limited to a computing device 202, a wireless transmission unit 318, a processing unit 306, a cloud 204, algorithms, and the like, some of which are described further in various embodiments described in more detail below.

Various embodiments of the present invention may include an external sensor module 200 for measuring the elderly's external body movement. Many types of external sensor modules 200 are known by those skilled in the art for measuring external body movement. These external sensor modules 200 include but are not limited to accelerometers and gyroscopes, combinations thereof, and the like. The part of the body wearing the external sensor module 200 and being measured for movement may be a limb, such as a wrist, ankle, heel, or finger or the trunk of the body, such as a shoulder, or a waist. In most embodiments, the use of a combination of 3D accelerometer and 3D gyroscope is preferably being used at the wrist of the elderly.

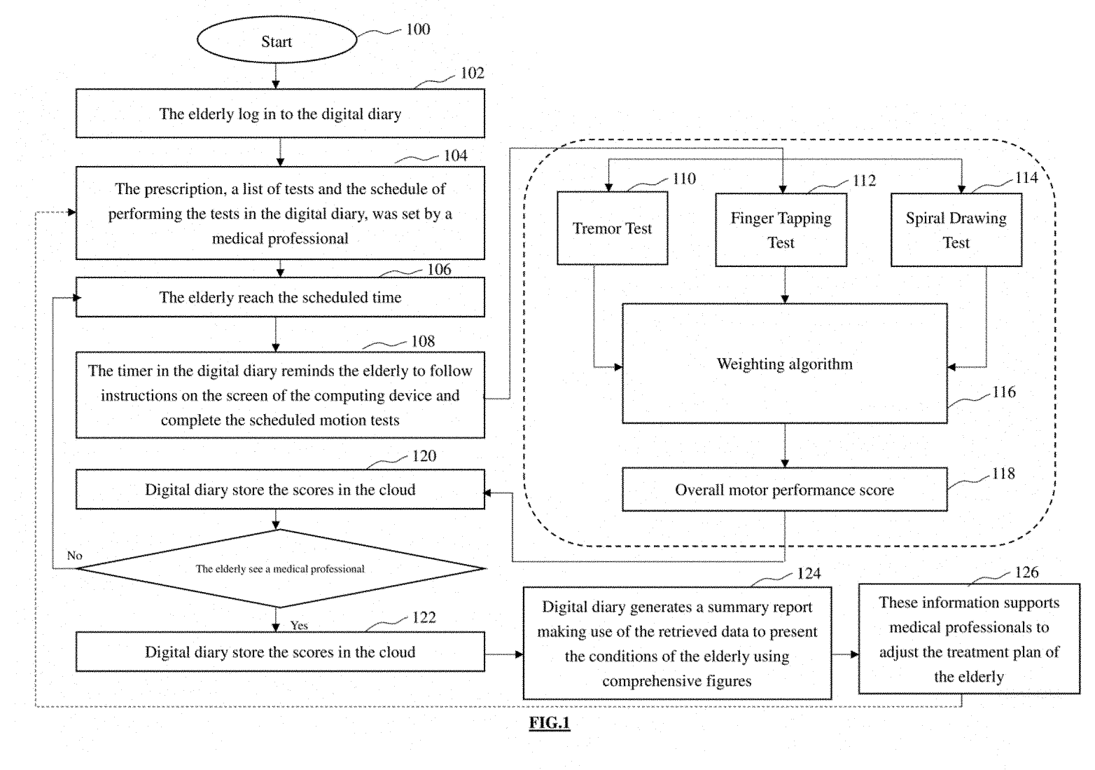

FIG. 1 is a flowchart illustration showing a method of monitoring and quantifying various symptoms of movement disorders for monitoring and treatment support for medical professionals. To initiate the monitoring and treatment support system for the elderly with movement disorders 100, the elderly need to download a mobile application 206 to their own personal computing devices 202. The computing device 202 includes but is not limited to a smartphone, a digitized tablet, and the like. The mobile application 206 is a digital diary for the elderly to track and record symptoms of movement disorders on a daily basis. The mobile application 206 is also designed for medical professionals as monitoring and treatment support platform to review and monitor the elderly's condition outside the clinical environment. The elderly then needs to get registered and log in to the digital diary 102.

To start using the digital diary 100, the elderly are required to input basic personal information such as Name, Age, Contact, Identity Card Number, Address, and the like, and as well as past clinical records such as the number of suffered years from movement disorders and the severity of the movement disorders, based on the standard clinical evaluation. The standard in evaluating the severity of movement disorders symptoms in Parkinson's disease is the Unified Parkinson's Disease Rating Scale (UPDRS). With the completion of all information as required in the digital diary, the elderly can therefore get registered and log in to the digital diary 102.

With registration to the digital diary, information will be stored in a cloud 204. The medical professional in charge can therefore assess the information provided by the elderly from the cloud 204 using the mobile application 206 which acts as a monitoring and treatment support platform. Medical professionals can also make up and give the elderly prescriptions through the monitoring and treatment support platform 104. To write out prescriptions for the elderly, the medical professionals can select the medicines from a medicine list in the monitoring and treatment support platform. Medical professionals can then adjust the quantity and the frequency of taking each medicine. Medical professionals can further set the start date and the end date of the prescriptions so that the elderly can directly refer to the digital diary of the details of their medications 104. Medical professionals can also arrange a schedule for the elderly to take medicines and carry out various motion tests 104. The schedule of motion tests varies with the elderlies, depending on their health conditions and the severity as assessed by medical professionals. The prescriptions and the schedule of the motion tests are stored in the cloud 204 of the system.

Whenever a scheduled time is reached 106, a timer in the digital diary would remind the elderly by sending notifications 108. The timer provides two types of reminders, medication reminder and test reminder. For medication reminder, the elderly need to follow the instructions and take medicines according to the quantity of the medicines as shown on the screen of the personal computing device 202 such as a digitized tablet. For test reminder, the elderly are required to complete the scheduled tests. The motion tests are scheduled for monitoring and quantifying symptoms of movement disorders. They are also scheduled for early detection of movement disorders. The motion tests are designed for symptoms include, but are not limited to, tremor, bradykinesia, rigidity, balance and dyskinesia. Preferably, motion tests developed in the present invention include tremor test 110, finger tapping test 112 and spiral drawing test 114 for quantifying symptoms of tremor, bradykinesia and dyskinesia respectively.

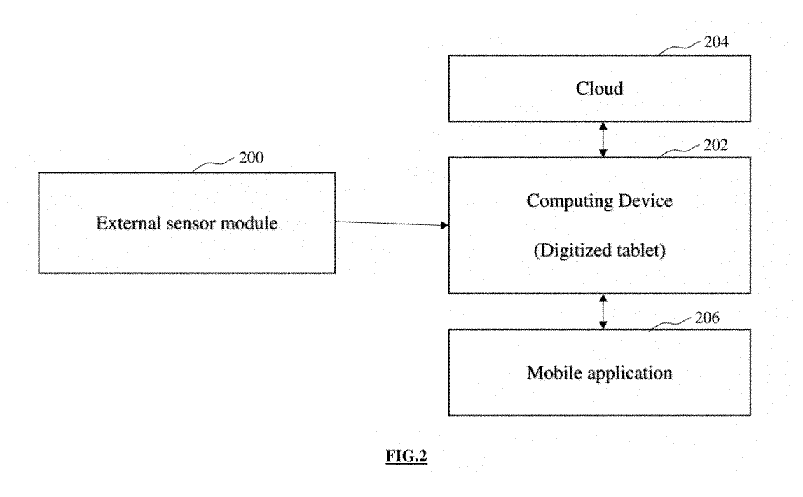

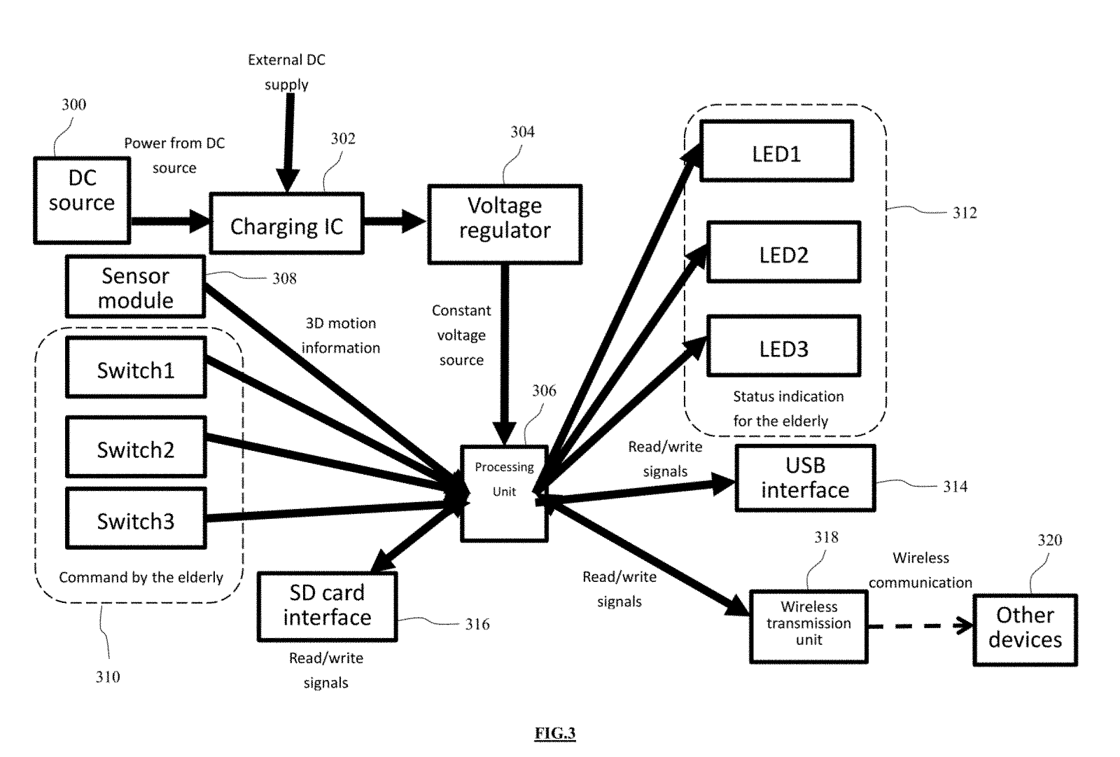

Referring to FIG. 2, at least one external sensor module 200, a computing device 202 such as a digitized tablet and a cloud 204 are required to conduct a tremor test 110. According to the one embodiment of the tremor test 110, the external sensor module 200 includes the components and interconnections detailed in FIG. 3: a processing unit 306, a sensor module 308, a wireless transmission unit 318, a data storage module and a power module. The sensor module 308 in FIG. 3 includes but are not limited to accelerometers and gyroscopes, combinations thereof, and the like. The sensor module 308 can be a 9-axis sensor. Many types of sensor modules 308 are known by those skilled in the art for measuring external body movement. The power module in FIG. 3 contains a DC source 300, a charging IC 302 and a voltage regulator 304. The charging IC 302 receives power from the DC source 300 in the external sensor module 200, and as well as from the external DC supply. Before supplying the power stored in the charging IC 302 to the processing unit 306, the power first passes through the voltage regulator 304 for power regulation. The voltage regulator 304 is configured to regulate the power coming from the charging IC 302. It prepares the power of the charging IC 302 for use by the processing unit 306. The data storage module in FIG. 3 contains a SD card interface 316 and a USB interface 314. Both SD card interface 316 and USB interface 314 are configured to read and/or write signals from and/or to the processing unit 306. The wireless transmission unit 318 is capable of communicating with other devices 320, for example, the computing device 202 such as a digitized tablet at the present invention. Moreover, the external sensor module 200 further consists of at least one switch 310 commanded by the elderly and at least one LED light 312 for indicating the status of the measurement to the elderly.

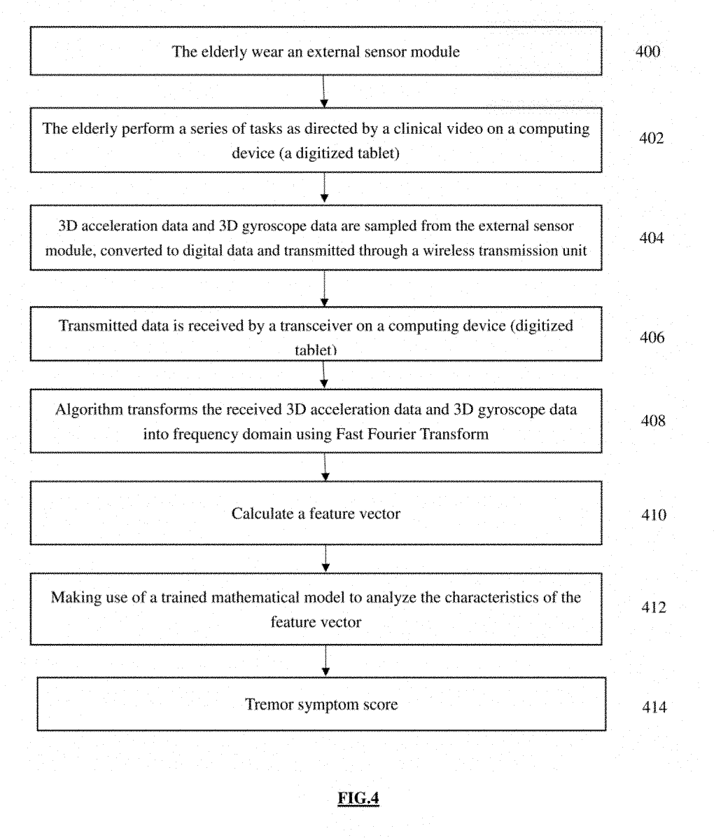

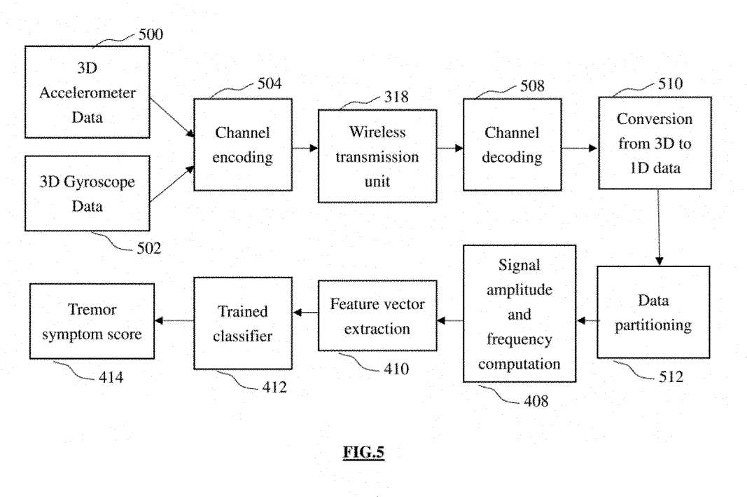

Referring now to FIGS. 4 and 5, upon receiving a test reminder for tremor test 110, the elderly should follow the instructions on the screen of the computing device 202 such as a digitized tablet. The elderly are instructed to wear at least one external sensor module 400. The part of the body wearing the external sensor module 200 and being measured for movement may be a limb, such as a wrist, ankle, heel, or finger or the trunk of the body, such as a shoulder, or a waist. In this embodiment, the use of a combination of 3D accelerometer and 3D gyroscope is preferably used at the wrist of the elderly. After wearing the external sensor module 200, the elderly can then use the computing device 202 such as a digitized tablet to perform a series of tasks as demonstrated in a clinical video 402. The elderly are instructed to hold at least one arm straight forward (keeping the arms perpendicular to the body). During the time, the external sensor module 200 captures and samples the 3D acceleration data 500 and 3D gyroscope data 502 from the elderly. The sampled 3D acceleration data 500 and 3D gyroscope data 502 are then converted to digital data for transmission 404. The digital data undergoes channel encoding 504 before it is transmitted through a wireless transmission unit 318. The digital data then goes through channel decoding 508 for further conversion of 3D data into 1D data 510. The transceiver in the computing device 202 such as a digitized tablet receives the transmitted data 406 in the form of 1D digital data from the external sensor module 200. An algorithm is developed to figure out the severity of tremor by transforming the digital acceleration and gyroscope data into a frequency domain using Fast Fourier Transform 408. Frequency amplitude of accelerometer and gyroscope signals are preferably the feature vectors for the tremor test 110. A trained algorithm stored in the cloud 204 is then used to calculate the feature vectors 410. Further, a trained mathematical model is used to analyze the characteristics of the feature vectors 412. Preferably, the trained mathematical models are kernel principal component analysis (KPCA) and/or kernel discriminant analysis (KDA) or other contemporarily available analysis for dimensionality reduction or for further feature extraction. A tremor symptom score 414 is therefore generated with comprehensive analysis on the characteristics of the feature vectors for reviewing the severity of tremor and stored in the cloud 204.

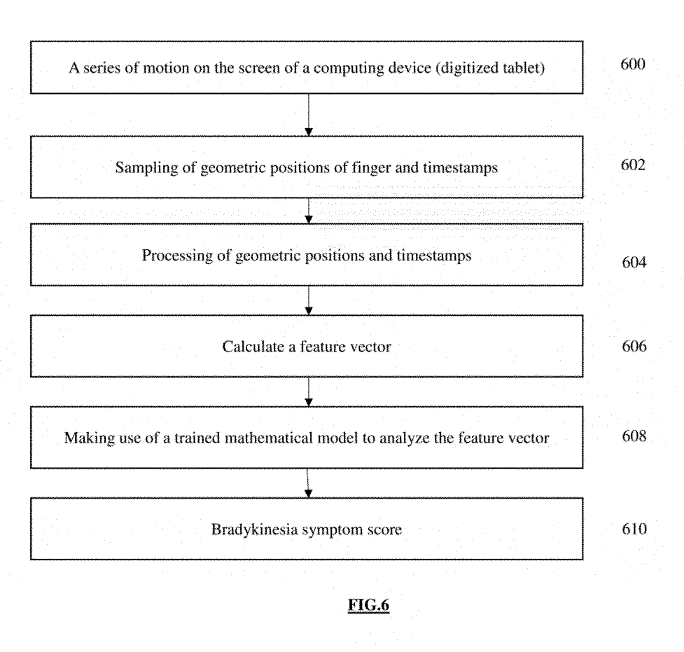

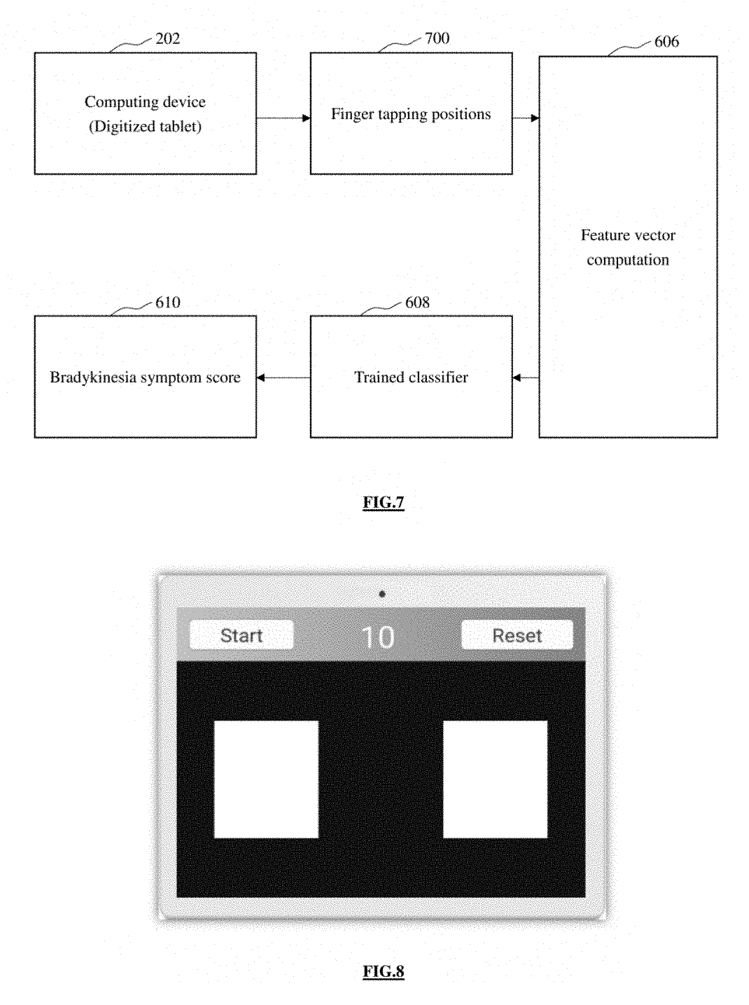

Referring to FIGS. 6 and 7, a method of performing finger tapping test 112 is shown for detecting the severity of bradykinesia. Upon receiving a test reminder for finger tapping test 112, the elderly should follow the instruction on the screen of the personal computing device 202 such as a digitized tablet. The elderly is instructed to perform a series of motions on the screen of the computing device 202 such as a digitized tablet 600. On the screen of the computing device 202 such as a digitized tablet, two squares are displayed, as shown in FIG. 8. The elderly are asked to alternately tap each side of the squares at the fastest speed for ten seconds. Preferably, the tapping motions are performed using a finger. The finger tapping task will repeat three times for each hand. During the time, the geometric positions of the finger and timestamps are sampled 602 for further processing 604 and the feature vectors are calculated 606. The feature vectors include but are not limited to the total distance of the finger movement, total dwelling time (which is the time lapse between two consecutive taps), instantaneous tapping speed of each movement and the tapping error (which occurred when the elderly tapped outside the squares). Further, a trained mathematical model is used to analyze the characteristics of the feature vectors 608. Preferably, the trained mathematical models are kernel principal component analysis (KPCA) and/or kernel discriminant analysis (KDA) or other contemporarily available analysis for dimensionality reduction or for further feature extraction. A bradykinesia symptom score 610 is therefore generated with comprehensive analysis on the characteristics of the feature vectors for reviewing the severity of bradykinesia and stored in the cloud 204.

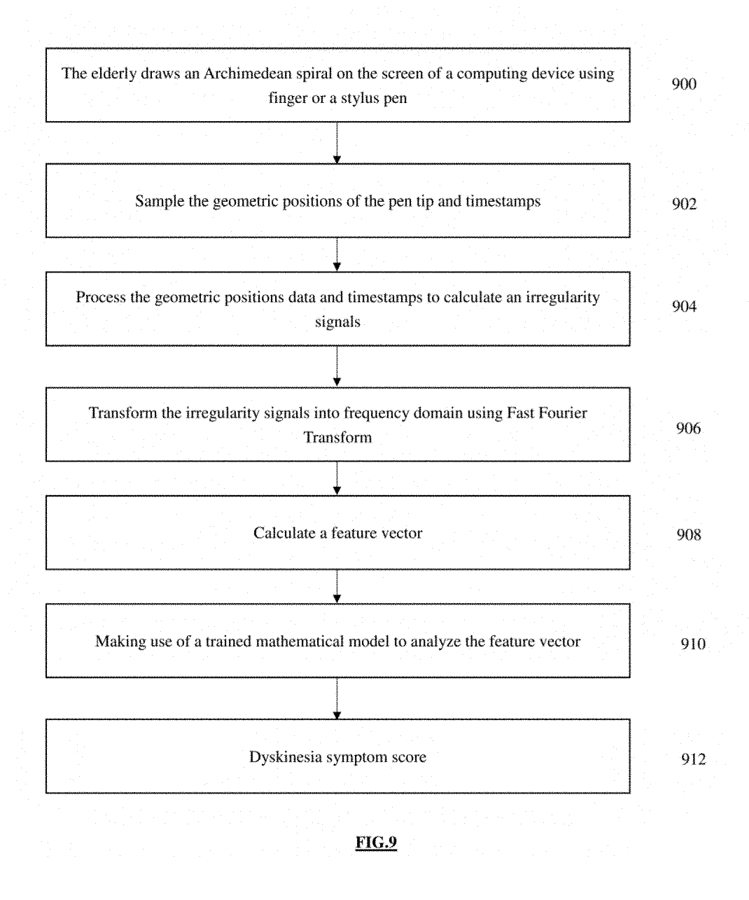

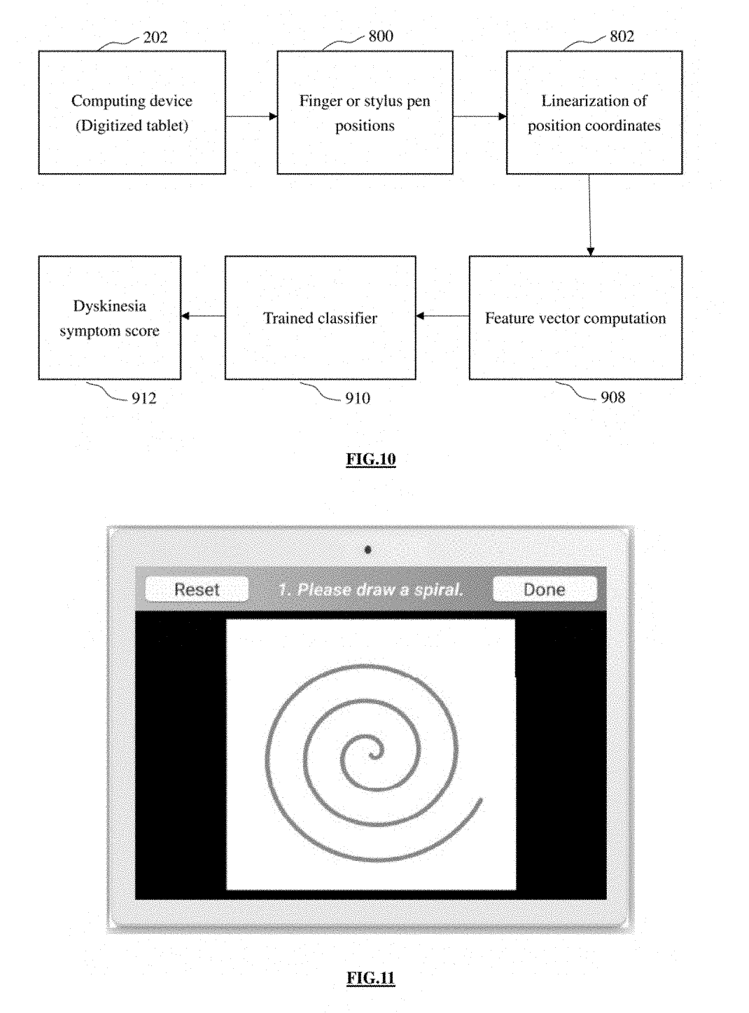

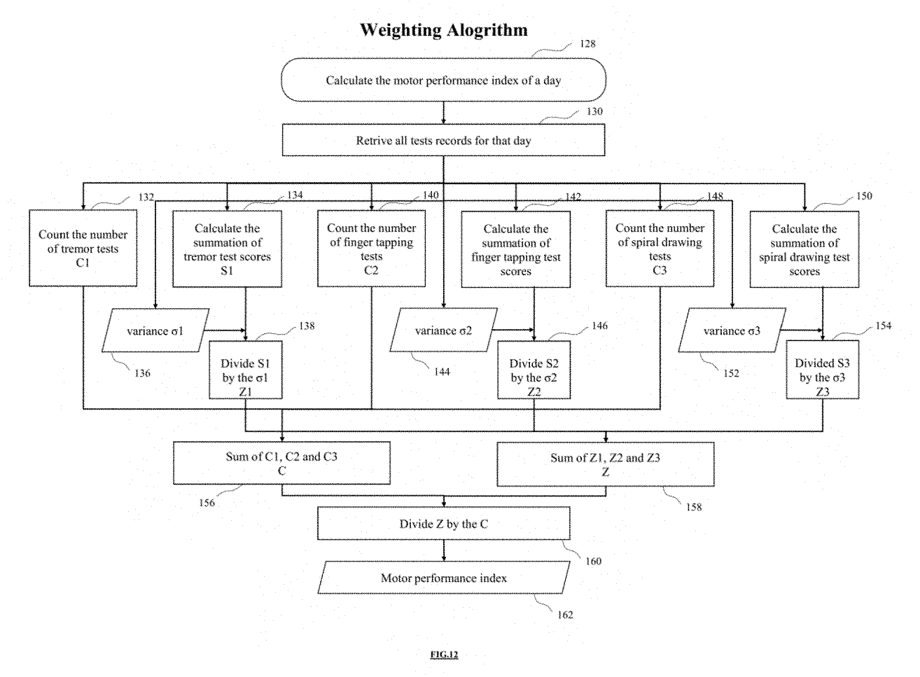

Referring to FIGS. 9 and 10, a method of performing spiral drawing test 114 is shown for detecting the severity of dyskinesia. Upon receiving a test reminder for spiral drawing test 114, the elderly should follow the instructions on the screen of the computing device 202 such as a digitized tablet. The elderly are instructed to perform a series of motions on the screen of the computing device 202 such as a digitized tablet. On the screen of the computing device 202, a spiral pattern is displayed, as shown in FIG. 11. In a preferred embodiment of the present invention, the spiral pattern is an Archimedean spiral pattern. The elderly are asked to trace the spiral pattern at a self-paced velocity. The spiral pattern can be drawn on the screen of the computing device 202 such as a digitized tablet using a finger or a stylus pen 900. In a preferred embodiment, the drawing motion is performed using a stylus pen. Each drawing should be performed from the center outwards and then inwards back to the center. The drawing is performed in both clockwise and anti-clockwise directions. During the time, the geometric positions of the pen tip or the finger and timestamps are sampled 902 for further processing to calculate irregularity signals 904. The irregularity signals are transformed into frequency domain using Fast Fourier Transform 906. Data in the frequency domain is then used for feature vectors computation 908. Preferably, the feature vector for spiral drawing test 114 is the frequency amplitude of the rate of instantaneous velocity change of drawing. Further, a trained mathematical model is used to analyze the characteristics of the feature vectors 910. Preferably, the trained mathematical models are kernel principal component analysis (KPCA) and/or kernel discriminant analysis (KDA) or other contemporarily available analysis for dimensionality reduction or for further feature extraction. A dyskinesia symptom score 912 is therefore generated with comprehensive analysis on the characteristics of the feature vectors reviewing the severity of dyskinesia and stored in the cloud 204.

With the completion of two or more motion tests to quantify symptoms of movement disorders, two or more scores from two or more motion tests will be further processed. In a preferred embodiment, scores from tremors test 110, finger tapping test 112 and spiral drawing test 114 are further processed using a weighting algorithm 116.

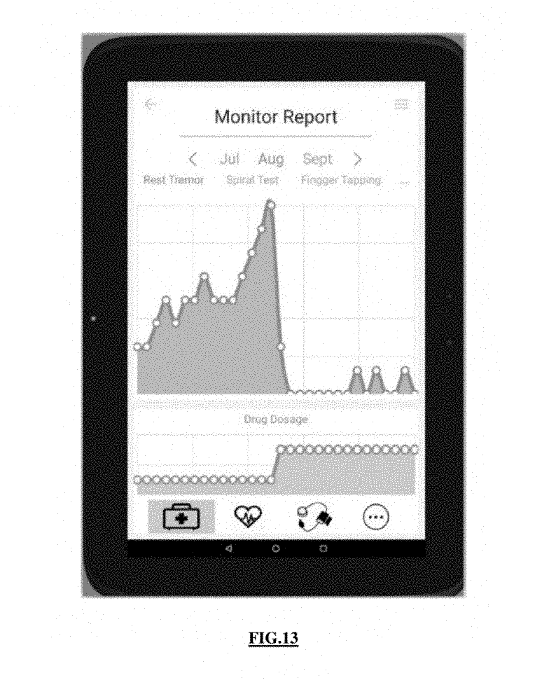

In the weighting algorithm 116, different weights are assigned to the scores derived from the tremor test 110, finger tapping test 112 and spiral drawing tests 114. Coefficients regarding the respective weights are applied to various scores from motion tests to generate an overall motor performance score 118. The weighting coefficients are with respect to the number motion test scheduled to be completed each day. Referring to FIG. 12, details of the weighting algorithm 116 are shown. To initiate the calculation of the motor performance index 162, which is also known as the overall motor performance score 118, the weighting algorithm 116 will first retrieve the record of the motion tests 130 from the cloud 204. The records include tremor symptom scores 414, bradykinesia symptom scores 610 and/or dyskinesia symptom scores 912 of each test and the number of tremor tests (C1) 132, the number of finger tapping tests (C2) 140 and/or the number of spiral drawing tests (C3) 148 conducted. The tremor symptom scores 414, bradykinesia symptom scores 610 and dyskinesia symptom scores 912 of each test are further processed and calculated as the summation of tremor symptom scores (S1) 134, summation of bradykinesia symptom scores (S2) 142 and summation of dyskinesia symptom scores (S3) 150 respectively. A variance of tremor symptom scores (σ1) 136, a variance of bradykinesia symptom scores (σ2) 144 and a variance of dyskinesia symptom scores (σ3) 152 are computed using the number of tests conducted 132, 140, 148 and the summation of scores 134, 142, 150 for further calculation. With the summation of scores 134, 142, 150 and variance 136, 144, 152 of each motion test, featuring indices 138, 146, 154 are obtained. The featuring index for tremor symptom (Z1) 138 is determined by dividing the summation of tremor symptom scores (S1) 134 by variance of tremor symptom (σ1) 132. Similarly, the featuring index for bradykinesia symptom (Z2) 146 is determined by dividing the summation of bradykinesia symptom scores (S2) 142 by variance of bradykinesia symptom (σ2) 144, and the featuring index for dyskinesia symptom (Z3) 154 is determined by dividing the summation of dyskinesia symptom scores (S3) 150 by variance of dyskinesia symptom (σ3) 152. With the sum of C1, C2 and C3 (C) 156 and the sum of Z1, Z2 and Z3 (Z) 158, the motor performance index 162 is therefore obtained by dividing Z by C 160. The equation of the motor performance index (MPI) 162 is shown as below:

$$MPI = \frac{{\sum }_{i = 1}^{N} ( \frac{{\sum }_{j}^{{C}_{i}} {s}_{i , j}}{{σ}_{i}} )}{{\sum }_{i = 1}^{N} {C}_{i}}$$

where,
N=the total number of test types provided by the system
Cᵢ=the number of times of the particular test scheduled to be completed for each day
σᵢ=the variance of scores of the particular testᵢ, which is extracted from the clinical data set.
s_{i,j}=the j-th test score of the particular testᵢ for that day

In this way, the motor performance index 162 can reflect the general condition of the elderly with movement disorders with only one indicator, instead of the performance on one specific motion test. The elderly also find the overall motor performance score 118 easier to understand as compared to the nonlinearity in inter- and intra-test scores. An overall motor performance score 118 can better demonstrate the severity of the elderly's movement disorders by weighting and by combining various motion tests that quantify symptoms of movement disorders into a single indicator.

The overall motor performance score 118 obtained from the weighting algorithm 116 is stored in a cloud 204. The cloud 204 is capable of storing more than one computed scores from more than one motion tests. In a preferred embodiment of the present invention, the cloud 204 can store the computed scores from tremor test 110, finger tapping test 112 and/or spiral drawing test 114. The cloud 204 of the present invention is also capable of storing trained models for analyzing and computing scores based on the movement data acquired from the motion tests. Whenever movement data is captured from the motion tests, the computing device 202 such as a digitized tablet will retrieve trained models from the cloud 204 and automatically select the particular trained model for calculating the respective feature vectors and hence deriving a score to reflect the severity of a symptom.

The overall motor performance score 118 stored in the cloud 204 is further analyzed. For the elderly who are classified mild movement disorders, they are not required to see a medical professional. They then keep following the schedule as arranged by a medical professional. The timer in the digital diary will remind the elderly at the scheduled time to perform the motion tests or to take medicines by sending notifications. For the elderly who reach a score indicating severe movement disorders, the elderly are recommended to see a medical professional. The medical professional in charge can therefore retrieve the elderly's information such as personal information and movement data related to movement disorders from the cloud 204 through the monitoring and treatment support platform.

Referring now to FIG. 13, movement data and the overall motor performance score 118 stored in the cloud 204 are used to generate a summary report in the monitoring and treatment support platform for medical professionals' review 124. The summary report is preferably presented in forms of comprehensive figures. The overall motor performance score 118 is depicted as a graph indicating the severity of the movement disorder. The treatment plan includes, but are not limited to, the amount of drug dosage and types and combinations thereof of drugs, is also depicted as a graph as a comparison with the severity of movement disorders. A medical professional can identify the efficacy of a prescription by comparing the severity of movement disorders with the treatment plan. Hence, the information provided in the summary report can support medical professionals when formulating and adjusting treatment plans to the elderly 126. Presenting the summary report in terms of figures is more user-friendly. The elderly can directly refer to the comprehensive figures to know more about their own health conditions. When the elderly are detected with abnormality in movement, they could find medical professionals for further professional judgement to determine whether they are suffered from movement disorders. The early detection of movement disorders with the help of the present invention improves the efficacy in treating movement disorders. Medical professionals can also directly refer to the comprehensive figures and give feedback to the elderly instead of analyzing on raw statistics. This saves time for medical professionals and avoids errors of subjective judgement by medical professionals. Medical professionals can instead write up a new prescription and test schedule for the elderly based on the summary report.

I claim:

1. A system for monitoring and quantifying symptoms of movement disorders for elderly care comprising:
at least one measuring device for acquiring movement data;
a computing device for displaying clinical videos and displaying analysis results;
a mobile application installed in the said computing device, wherein a timer is enclosed in the mobile application for sending notifications and reminding the elderly at scheduled time as arranged by medical professionals;
a processing unit in the said measuring devices with at least one trained algorithm to calculate scores of each motion test; and
a cloud for storing at least one computed score that reveals symptoms of movement disorders and storing trained models;

2. The system of claim 1, wherein the said measuring device and the said computing device can be a digitized tablet.

3. The system of claim 2, wherein the said digitized tablet comprises means for displaying instructions utilizing clinical videos and displaying clinical results.

4. The system of claim 2, wherein the said digitized tablet comprises means for sampling geometric positions and timestamps in finger tapping test and sampling geometric positions and timestamps in spiral drawing test.

5. The system of claim 1, wherein the said mobile application is a digital diary for the elderly.

6. The system of claim 5, wherein the said digital diary comprises:
means for recording symptoms of movement disorders using the said computing device;
means for reminding patients at scheduled time to take medications or perform motion tests by the said timer;
means for calculating an overall motor performance score using a weighting algorithm; and
means for storing the said overall motor performance score in the said cloud.

7. The system of claim 6, wherein the said timer is pre-set by medical professionals based on the elderly' health conditions.

8. The system of claim 7, wherein the said timer alerts the elderly by providing medication reminder and test reminder.

9. The system of claim 6, wherein the said weighting algorithm computes at least one featuring index by dividing the summation of scores of one motion test by a variance of one motion test for calculating the said overall motor performance score.

10. The system of claim 1, wherein the said mobile application is a monitoring and treatment support platform for medical professionals.

11. The system of claim 10, wherein the said monitoring and treatment support platform comprises:
means for assessing personal information and clinical data of the elderly;
means for writing up prescriptions and scheduling tests for the elderly; and
means for generating a summary report based on recorded movement data from the said cloud in forms of comprehensive figures.

12. The system of claim 11, wherein the said means for writing up prescriptions further comprises means for making adjustment to the said prescriptions.

13. The system of claim 11, wherein the said summary report is presented in graphs, representing the severity of movement disorders with reference to the said overall motor performance score.

14. The system of claim 13, wherein the said graphs for demonstrating the severity of movement disorders are compared with the treatment plan including the amount of drug dosage and types and combinations thereof of drugs.

15. The system of claim 14, wherein the said graphs are adopted to provide medical professionals comprehensive report with reference to the said treatment plan to determine the efficacy of treatment.

16. The system of claim 1, wherein the said trained algorithm quantifies the severity of tremor using:
frequency amplitude of accelerometer and gyroscope signals as feature vectors; and
kernel principal component analysis (KPCA) or kernel discriminant analysis (KDA) for dimensionality reduction or further feature extraction.

17. The system of claim 1, wherein the said trained algorithm quantifies the severity of bradykinesia using:
total distance of finger movement, total dwelling time, instantaneous tapping speed of each movement and tapping error as feature vectors; and
kernel principal component analysis (KPCA) or kernel discriminant analysis (KDA) for dimensionality reduction or further feature extraction.

18. The system of claim 1, wherein the said trained algorithm quantifies the severity of dyskinesia using:
frequency amplitude of rate of instantaneous velocity change of drawing as feature vector; and
kernel principal component analysis (KPCA) or kernel discriminant analysis (KDA) for dimensionality reduction or further feature extraction.

19. The system of claim 1, wherein the said measuring device further comprises an external sensor module with a combination of 3D accelerometer and 3D gyroscope.

20. The system of claim 19, wherein the said external sensor module comprises means for sampling acceleration data and gyroscope data in tremor test.

21. A method for monitoring and quantifying symptoms of movement disorders for elderly care comprising the steps of:
instructing the elderly using clinical videos and displaying interfaces for motion tests;
obtaining movement data from at least one measuring device;
providing a mobile application installed in a computing device, wherein a timer is embedded in the mobile application for sending notifications and reminding the elderly at scheduled time as arranged by medical professionals;
generating one or more scores representing the severity of one or more symptoms of movement disorders;
processing the said scores to derive an overall motor performance score as an indicator for the severity of movement disorders using a weighting algorithm; and
storing the said overall motor performance score in a cloud.

22. The method of claim 21, wherein the said step of obtaining movement data from the said measuring device further comprises the step of obtaining movement data from the said computing device.

23. The method of claim 22, wherein the said step of obtaining movement data from the said computing device further comprises the step of sampling geometric positions and timestamps in finger tapping test and sampling geometric positions and timestamps in spiral drawing test.

24. The method of claim 21, wherein the step of providing the said mobile application further comprises the step of providing a digital diary for the elderly.

25. The method of claim 24, wherein the said step of providing the said digital diary for the elderly further comprising:
inputting personal information and symptoms of movement disorders;
providing the said timer to remind the elderly to take medications and perform motion test at scheduled time; and
generating one or more scores representing the severity of one or more symptoms of movement disorders.

26. The method of claim 25, wherein the said step of providing the said timer further comprises the step of pre-setting the said timer by medical professionals based on the elderly's health conditions.

27. The method of claim 26, wherein the said step of providing the said timer further comprises the step of providing medication reminder and test reminder.

28. The method of claim 25, wherein the said step of generating one or more scores further comprising:
computing a score from tremor test;
computing a score from finger tapping test; and
computing a score from spiral drawing test.

29. The method of claim 28, wherein the said step of generating one or more scores further comprises the step of processing the said scores to derive the said overall motor performance score as an indicator for the severity of movement disorders using the said weighting algorithm.

30. The method of claim 29, wherein the said weighting algorithm comprises the steps of:
retrieving record of at least one motion test, including the number of test conducted and the said scores for each motion test;
computing the summation of scores of each motion test;
computing a featuring index using the summation of scores of each motion test and a variance of each motion test; and
computing the said overall motor performance score by dividing the said feature indices from at least one motion test by the number of motion tests conducted.

31. The method of claim 30, wherein the said featuring index is computed by dividing the summation of motion test scores by the said variance of the respective motion test.

32. The method of claim 29, wherein the said step of deriving the said overall motor performance score further comprises the step of storing the said overall motor performance score in the said cloud.

33. The method of claim 21, wherein the said step of providing a mobile application further comprises the step of providing a monitoring and support platform for medical professionals.

34. The method of claim 33, wherein the step of providing a monitoring and support platform further comprising:
assessing the elderly's information and movement data from the said cloud;
making up prescriptions and scheduling motion tests for the elderly;
presenting a summary report using movement data stored in the said cloud.

35. The method of claim 34, wherein the said step of making up prescriptions for the elderly further comprises the step of making adjustments to the said prescriptions.

36. The method of claim 34, wherein the said step of presenting the said summary report further comprises the step of presenting the said summary report in graphs for representing the severity of movement disorders with reference to the said overall motor performance score.

37. The system of claim 36, wherein the said step of presenting the said summary report in graphs is adopted to compare with the said treatment plan including the amount of drug dosage and types and combinations thereof of drugs.

38. The method of claim 21, wherein the said step of obtaining movement data from the said measuring device further comprises the step of obtaining movement data from an external sensor module.

39. The method of claim 38, wherein the said step of obtaining movement data from the said external sensor module further comprises the step of sampling acceleration data and gyroscope data in tremor test.
